(19) United States
(12) Patent Application Publication
(10) Pub. No.: US 2026/0265191 A1
(43) Pub. Date: Sep. 10, 2026

(54) PRODUCING 2,5-FURANDICARBOXYLIC ACID AND TREATING OVERHEAD STREAM

(71) Applicant: Furanix Technologies B.V., Amsterdam (NL)

(72) Inventors: Jeffrey John Kolstad, Amsterdam (NL); Jan Hendrik Blank, Amsterdam (NL); Gerardus Johannes Maria Gruter, Amsterdam (NL)

(21) Appl. No.: 18/871,644

(22) PCT Filed: Jun. 13, 2023
(86) PCT No.: PCT/EP2023/065709

§ 371 Date: Dec. 4, 2024

(30) Foreign Application Priority Data
Jun. 14, 2022 (EP) 22178818.5

(51) Int. Cl. C07D 207/34

(52) U.S. Cl. CPC C07D 207/34

(57) ABSTRACT

Process for producing 2,5-furandicarboxylic acid, comprising the steps of: a) oxidizing feed comprising 5-methylfurfural using an oxidizing gas at a temperature in the range of 150 to 210° C. in the presence of a catalyst system comprising cobalt, manganese and bromine to obtain a reaction mixture; b) separating from the reaction mixture obtained in step a) an overhead stream and crude carboxylic acid composition comprising 2,5-furandicarboxylic acid; and c) treating at least part of the overhead stream with base wherein the amount of base is at least of from 0.7 to 3 times, on an equinormal basis, of the amount of bromide in the overhead stream to be treated.

### TECHNICAL FIELD

The present invention relates to a process for producing 2,5-furandicarboxylic acid.

### BACKGROUND

2,5-Furandicarboxylic acid (FDCA) is known in the art to be a highly promising building block for replacing petroleum-based monomers in the production of high performance polymers. In recent years, 2,5-furandicarboxylic acid and the novel plant-based polyester polyethylenefuranoate (PEF), a completely recyclable plastic with superior performance properties compared to today's widely used petroleum-based plastics, have attracted a lot of attention. These materials could provide a significant contribution to reducing the dependence on petroleum-based polymers and plastics, while at the same time allowing for a more sustainable management of global resources. Comprehensive research was conducted in the field to arrive at a technology for producing 2,5-furandicarboxylic acid and PEF in a commercially viable way.

2,5-Furandicarboxylic acid is typically obtained by oxidation of molecules having furan moieties, e.g. 5-hydroxymethylfurfural (5-HMF) as well as the corresponding esters and ethers, e.g. 5-alkoxymethylfurfural, and similar starting materials, that are typically obtained from plant-based sugars, e.g. by sugar dehydration. A broad variety of oxidation processes is known from the prior art such as enzymatic and metal catalysed processes, either heterogeneous or homogeneous. WO 2014/014981 and WO 2011/043661 describe processes using catalyst systems comprising cobalt, manganese and bromine to oxidize compounds having a furan moiety to 2,5-furandicarboxylic acid using oxygen or air as an oxidizing agent.

It was found that oxidation of compounds containing furan moieties produced overhead streams containing a substantial amount of bromide. Such overhead streams were found to cause corrosion thereby increasing the demand on materials of construction and preventing retrofit opportunities.

Surprisingly, it now has been found that feed comprising 5-methylfurfural (5-MF) produced overhead streams having a reduced bromide content.

WO2012/161968 describes oxidation of a wide range of furan containing compounds. Examples 11a and 11b of WO2012/161968 oxidize 5-methylfurfural at a temperature of 130° C. The amount of bromide in the overhead stream is not disclosed.

While oxidation of compounds such as 5-hydroxymethylfurfural and ethers thereof has been extensively studied, less is known about oxidation of 5-methylfurfural. Soviet Union Inventor's Certificate 441877 describes the conversion of 5-methylfurfural into 2,5-furandicarboxylic acid. WO2011043661 describes 5-methylfurfural as a possible feed for oxidation. Neither document describes the composition of overhead streams.

### DISCLOSURE OF THE INVENTION

It was an objective to reduce the bromide content of side streams, especially overhead streams, obtained in the oxidation of oxidizable compounds to produce 2,5-furandicarboxylic acid.

Surprisingly, it was found that the use of 5-methylfurfural as oxidizable feed produced overhead streams having reduced bromide content. Subsequent neutralization of bromide further reduces the risk of corrosion.

The invention relates to a process for producing 2,5-furandicarboxylic acid, comprising the steps of: a) oxidizing feed comprising 5-methylfurfural using an oxidizing gas at a temperature in the range of 150 to 210° C. in the presence of a catalyst system comprising cobalt, manganese and bromine to obtain a reaction mixture; b) separating from the reaction mixture obtained in step a) an overhead stream and crude carboxylic acid composition comprising 2,5-furandicarboxylic acid; and c) treating at least part of the overhead stream with base wherein the amount of base is at least of from 0.7 to 3 times, on an equinormal basis, the amount of bromide in the overhead stream to be treated.

The amount of bromide is determined by potentiometric titration with silver nitrate solution. Potentiometric titration is well known and details are described in analytical handbooks such as the article Titrimetry, Potentiometric by A. Hulanicki and S. Glab, pages 114-121 of the Encyclopedia of Analytical Science (Second Edition), 2005.

The expression bromide is used hereinafter to indicate the content of free bromine ions determined to be present by this titration method.

Equinormal means the same number of moles of reactive units. In other words, equinormal means molarity times the number of reactive units. If the bromide is treated with a base containing a single hydroxide group, the molar amount of base is of from 0.7 to 3 times the molar amount of bromide. If the bromide is treated with a base containing two hydroxide groups, the molar amount of base is of from 0.35 to 1.5 times the molar amount of bromide.

The amount of bromide in the overhead stream can fluctuate. If so, the time averaged amount of bromide is to be used. If the operation of the process is continuous or semi-continuous, it is preferred that the average is determined on a regular basis, e.g. daily, and the amount of base is adjusted in accordance therewith.

Besides the overhead stream and the crude carboxylic acid composition comprising 2,5-furandicarboxylic acid, further side streams and/or product streams can be separated from the reaction mixture obtained in step a). Preferably, gaseous compounds are removed from the reaction mixture obtained in step a) as the overhead stream of step b) with the remainder being the crude carboxylic acid composition comprising 2,5-furandicarboxylic acid of step b).

### MODES FOR CARRYING OUT THE INVENTION

The process is aimed at producing 2,5-furandicarboxylic acid. The final product obtained tends to contain further compounds besides 2,5-furandicarboxylic acid especially if the final product obtained is the crude solid 2,5-furandicarboxylic acid produced in step b). The solid containing 2,5-furandicarboxylic acid obtained in step d), especially after having been washed and optionally further purified in any of steps e) to h) will contain a lower amount of further compounds besides 2,5-furandicarboxylic acid.

Step a) comprises oxidation of a feed comprising 5-methylfurfural. The feed comprises at least 5-methylfurfural optionally in combination with further compounds. The feed preferably comprises at least 80% by weight of 5-methylfurfural, more preferably at least 90% by weight of 5-methylfurfural, more preferably at least 95% by weight of 5-methylfurfural. The feed preferably consists of 5-methylfurfural.

Besides the feed, the mixture present in step a) comprises oxidizing gas, acetic acid and a catalyst system. After the reaction has started, the mixture present in step a) tends to further contain water produced by oxidation of 5-methylfurfural.

Step a) preferably is carried out at a temperature in the range of 150 to 210° C., preferably a temperature of 160 to 190° C., more preferably a temperature in the range of from 165 to 180° C. Preferably, the pressure in step a) is in the range of 700 to 2000 kPa. These parameters were found to produce 2,5-furandicarboxylic acid of good purity in good yields while at the same time enabling the reactors to be run such that the substantial heat generated by oxidation is removed by vaporization of a portion of the solvent. This is known in the art as adiabatic operation.

The catalyst system comprises cobalt, manganese and bromine either as the element or as a derivative thereof. The catalyst system preferably has a weight ratio of cobalt to manganese in the catalyst system of 10 or higher, preferably 15 or higher, and/or a weight ratio of bromine to the combined weight of cobalt and manganese in the catalyst system of 1 or higher, preferably 1.5 or higher, most preferably 2 or higher, wherein the value is preferably less than 4.0, more preferably less than 3.5. If the catalyst system comprises other metals besides cobalt and manganese in an amount of 5% by weight or more relative to the total amount of cobalt and manganese, it is preferred that the above ratios are achieved for the weight ratio of bromine to the combined weight of all metals in the catalyst system. The metals preferably are added as salts which are soluble in the reaction mixture. Typically, the amount of cobalt is selected in the range of 500 to 6000 ppm by weight, based on the weight of the feed, acetic acid and catalyst system. The amount of manganese typically is in the range from 20 to 6000 ppm by weight, based on the weight of the feed, acetic acid and catalyst system Typically, the bromine concentration would be from 30 to 8000, preferably 50 to 4500 ppm by weight of bromine, based on weight of the the feed, acetic acid and catalyst system. Alternatively, the bromine content is from 3000 to 8000 ppm by weight.

The oxidizing gas can be any gas known to be suitable by the person skilled in the art. Preferably, the oxidizing gas comprises molecular oxygen. Most preferably, the oxidizing gas is air.

The reactor for carrying out the oxidation can be any typical oxidation reactor that is known in the art.

After the reaction has started, the gas present tends to contain not only the oxidizing gas but also further compounds which have been vaporized due to the temperature increase caused by the oxidation. Therefore, the gas present after start of the oxidation tends to be a mixture of compounds. The mixture of compounds present as gas during the oxidation of step a), is referred to as overhead stream irrespective whether in the gaseous phase or subsequently brought in the liquid phase or mixture of gas and liquid. It will be clear that this overhead stream does not need to be removed overhead. The overhead stream can be separated from the reaction mixture in any way known to be suitable by the person skilled in the art.

The overhead stream can contain significant amounts of acetic acid, water and lesser amounts of organo-bromine compounds besides bromide containing compounds.

The overhead stream can be treated as such in step c). In a preferred embodiment, overhead stream separated in step b) is cooled to obtain a fluid which is at least partly liquid which fluid is the overhead stream treated in step c).

Preferably, acetic acid containing mixture is removed from the overhead stream which has been treated in step c). This acetic acid containing mixture subsequently is sent to step a). The mixture tends to contain further compounds such as water besides acetic acid. Preferably, the acetic acid containing mixture contains at least 90% by weight, more preferably at least 95% by weight of acetic acid. The acetic acid containing mixture preferably is separated by distillation from the overhead stream which has been treated in step c). Preferably, the acetic acid containing mixture has a higher concentration of acetic acid than the overhead stream originally obtained in step b).

A post-oxidation step has been found to be preferred especially when employed at high temperature. Most preferred is a process wherein a post oxidation step a1) is applied after step a) at a temperature in the range of 150 to 210° C., more specifically of 160 to 210° C.

At least part of the overhead stream obtained in step b) is treated with base in step c).

The overhead stream obtained in step b) preferably contains at most 800 parts per million by weight (ppmw) of bromide before being treated in step c), more preferably at most 500 ppmw, more specifically at most 400 ppmw.

The base specifically is selected from the group consisting of alkali metal hydroxides, alkaline earth metal hydroxides, alkali metal carbonates, alkaline earth metal carbonates, alkali metal bicarbonates, alkaline earth metal bicarbonates, ammonia, aqueous solutions of ammonia, organic derivatives of ammonia and aqueous solutions of organic derivatives of ammonia and mixtures thereof. Preferably, the base is selected from the group consisting of alkali metal hydroxides, alkaline earth metal hydroxides, alkali metal carbonates, alkaline earth metal carbonates and mixtures thereof, more preferably from the group consisting of alkali metal hydroxides, alkaline earth metal hydroxides and mixtures thereof.

The amount of base is of from 0.7 to 3 times, on an equinormal basis, of the amount of bromide in the overhead stream, more specifically at least 0.8 times, more specifically at least 1.0 or greater. It is preferred that the amount of base is not a large excess as that may hamper recovery of acetic acid in a subsequent step. The amount of base preferably is at most 2.0 times the amount of bromide, more specially at most 1.5 times the amount of bromide, on an equinormal basis.

The base is preferably added before overhead stream comes into contact with equipment such as overhead condensers under corrosive operating conditions.

The process preferably further comprises step d) separating the crude carboxylic acid composition in a separation zone to obtain solid containing 2,5-furandicarboxylic acid and effluent. Not all of the 2,5-furandicarboxylic acid generally will be removed from the crude carboxylic acid composition while generally not all of the solid which is separated will be 2,5-furandicarboxylic acid. Furthermore, it is preferred but not required to subject all crude carboxylic acid composition obtained in step d) to step e) and preferably steps f) tot h) all as discussed hereinafter.

Preferably, at least 50% by weight with respect to the weight of the dry crude solid 2,5-furandicarboxylic acid will be 2,5-furandicarboxylic acid, more preferably at least 70% by weight, more preferably at least 80% by weight, more preferably at least 90% by weight, more preferably at least 95% by weight, more preferably at least 98% by weight. Other compounds which can be present as part of the crude solid 2,5-furandicarboxylic acid are derivatives of 2,5-furandicarboxylic acid such as methyl ester of 2,5-furandicarboxylic acid, 5-hydroxymethyl-furan-2-carboxylic acid (HMFCA), 2-carboxy-5-(formyl) furan (FFCA), 5-bromo-2-furoic acid (Br-FCA) and bis-carbonyl-furoic acid (BCFCA).

Generally, the effluent obtained in step d) will be a liquid more especially mother liquor obtained after separating solid 2,5-furandicarboxylic acid in a solid-liquid separation zone. A preferred separation is by filtering or with the help of a centrifuge, more preferably a filter, more preferably a rotary pressure filter.

Different parts of the effluent can be subjected to different treatments.

Effluent can be treated to recover acetic acid. Generally, acetic acid containing mixture is removed from the effluent. The mixture tends to contain further compounds such as water besides acetic acid. Preferably, the acetic acid containing mixture contains at least 90% by weight, more preferably at least 95% by weight of acetic acid. The acetic acid containing mixture preferably is separated by distillation from the effluent. In a preferred embodiment, acetic acid containing mixture removed from effluent of step d) is sent to step a).

It is preferred that of from 60 to 99% by weight, more preferably of from 80 to 98% by weight, of the effluent is sent to step a) as so-called recycled mother liquor stream.

The process of the present invention shows its full potential in continuous or semi-continuous processes as it is especially desirable to prevent corrosion for such operation.

Preferred is a process according to the invention, wherein the process further comprises a step e) washing the crude solid 2,5-furandicarboxylic acid with a first washing solution comprising a saturated organic acid solvent having from 2 to 6 carbon atoms, preferably acetic acid, and less than 15%, preferably less than 10%, by weight of water, based on total washing solution. Such process step can be preferred to further reduce the amount of catalyst metals in the crude solid 2,5-furandicarboxylic acid. Preferred is a process wherein the crude solid 2,5-furandicarboxylic acid obtained is further washed with a second washing solution comprising water in an amount of more than 95%, preferably more than 99%, by weight with respect to the weight of the washing solution. This process step can further reduce the amount of manganese and/or cobalt.

In a preferred embodiment, the process further comprises f) contacting washed solid containing 2,5-furandicarboxylic acid obtained in step e) with polar solvent to obtain a solution; g) contacting the solution with hydrogen in the presence of a hydrogenation catalyst at hydrogenation conditions yielding a hydrogenated solution; and h) separating purified 2,5-furandicarboxylic acid from the hydrogenated solution, preferably separating by crystallization. Suitable process conditions are for example described in WO2016/195490. Preferred process conditions comprise contacting with hydrogen at a temperature in the range of 150 to 200° C. and a contact time with the hydrogenation catalyst in the range of 5 seconds to 15 min.

Step f) suitably comprises mixing the solid obtained in step e) with polar solvent to substantially fully dissolve the 2,5-furandicarboxylic acid and any further furan containing compounds. Preferably, the polar solvent is selected from the group consisting of water, acetic acid and mixtures thereof.

It will be clear to the person skilled in the art that preferably all solution is subjected to step g) although it is possible to use part of the solution only. Hereinafter, the invention is described in more detail using experiments.

### Example 1

The oxidation reactor is a 600 ml stirred pressure vessel, with two impellors. The reactor is pre-charged with a mixture having a total weight of 310 grams. The mixture comprises catalyst components provided as cobalt (II) acetate tetrahydrate, manganese (II) acetate tetrahydrate, and HBr as 48% by weight (wt %) in water. The amounts of the catalyst components are such as to yield a mixture which contained 3300 ppm Co, 188 ppm Mn and 7000 ppm Br. Water is added in an amount to result in 5 wt % of the total mixture, after accounting for the water introduced as part of the catalyst components. The balance is acetic acid.

The oxidation reactor is purged, pressurized, and heated to the desired operating temperature with stirring at 2000 rpm. The feed of Experiment 1 was 5-methoxymethylfurfural (MMF), the feed for Experiments 2, 3 and 4 was 5-methyl furfural (5-MF), the feed for Experiment 5 was a mixture of 5-methyl furfural (MF) and 5-methylmethoxy furfural (MMF) (weight ratio 70/30) and the feed of Experiment 6 was 5-hydroxymethyl-2-furaldehyde (5-HMF).

The process is started with a typical feed rate 8.3 mmol/minute. This feed rate was continued for 60 minutes (total feed 500 mmol) in the first set of experiments (Comparative Experiment 1 and Experiments 2 and 3) and for 30 minutes (total feed 250 mmol) in the second set of experiments (Experiments 4 and 5 and Comparative Experiment 6).

The oxidation reactor was purged, pressurized and heated to the desired operating temperature with stirring at 2000 rpm. A flow rate of lean air (8% oxygen) is started at a typical flow rate of 10 normal L/minute. The reaction typically begins within 3 minutes, noticed by a sharp decrease in oxygen in the outlet and an increase in CO and CO₂. During the reaction heat is generated, and a vapor stream is taken overhead and condensed. This vapor stream comprises mainly acetic acid and water. The amount of acetic acid solvent captured in the overhead is continuously monitored, and made up in the oxidation reactor with a fresh flow of acetic acid solvent to the reactor.

The typical operating pressure was 12 to 14 barg at 160° C. oxidation temperature.

At the end of the desired feed period, the feed of oxidizable compound is stopped, and the contents of the oxidation reaction is subjected to a period of post-oxidation.

The oxidation was followed by post-oxidation. Post-oxidation was conducted by stopping the flow of lean air for 1 minute and then re-establishing lean air flow at 4 NI/min for 20 minutes while maintaining the reaction temperature at 160° C.

Solids were separated by filtration and the cake obtained was washed twice with 1 part solvent (95 acetic acid to 5 parts water, by weight) to 1 part estimated dry cake weight each time.

The amount of bromide in the overhead stream was determined by potentiometric titration with silver nitrate solution.

The results are shown in the below table.

TABLE 1
Oxidation of feed

| | | Amount of | Bromide in | mg Br / |
|---|---|---|---|---|
| # | Feed | feed (mmol) | overhead (ppm) | mmol feed |
| Comp. | MMF | 500 | 1022 | 0.66 |
| Exp. 1 | | | | |
| Exp. 2 | MF | 500 | 141 | 0.08 |
| Exp. 3 | MF | 500 | 257 | 0.16 |
| Exp. 4 | MF | 250 | 208 | 0.14 |
| Exp. 5 | 70 MF / 30 MMF | 250 | 350 | 0.23 |
| Comp. | HMF | 250 | 1038 | 0.68 |
| Exp. 6 | | | | |

Table 1 shows that a feed comprising 5-methylfurfural or a mixture of 5-methylfurfural and alkoxymethyl-2,5-furfural resulted in an overhead stream containing a strongly reduced amount of bromide especially if the feed consisted of 5-methylfurfural.

1. A process for producing 2,5-furandicarboxylic acid, comprising the steps of:
a) oxidizing feed comprising 5-methylfurfural using an oxidizing gas at a temperature in the range of 150 to 210° C. in the presence of a catalyst system comprising cobalt, manganese and bromine to obtain a reaction mixture;
b) separating from the reaction mixture obtained in step a) an overhead stream and crude carboxylic acid composition comprising 2,5-furandicarboxylic acid; and
c) treating at least part of the overhead stream with base wherein the amount of base is at least of from 0.7 to 3 times, on an equinormal basis, of the amount of bromide in the overhead stream to be treated which amount of bromide is determined by potentiometric titration with silver nitrate solution.

2. The process according to claim 1 wherein the base is selected from the group consisting of alkali metal hydroxides, alkaline earth metal hydroxides, alkali metal carbonates, alkaline earth metal carbonates and mixtures thereof.

3. The process according to claim 1 wherein the amount of base is at least 1.0 or greater than the amount of bromide, on an equinormal basis.

4. The process according to claim 1 wherein the process further comprises cooling overhead stream separated in step b) to obtain a fluid which is at least partly liquid which fluid is the overhead stream treated in step c).

5. The process according to claim 1 wherein acetic acid containing mixture is removed from the overhead stream which has been treated in step c) which acetic acid containing mixture is sent to step a).

6. The process according to claim 1 wherein the process further comprises
d) separating the crude carboxylic acid composition comprising 2,5-furandicarboxylic acid in a separation zone to obtain solid containing 2,5-furandicarboxylic acid and effluent.

7. The process according to claim 1 wherein the feed consists of 5-methylfurfural.

8. The process according to claim 1, wherein the process further comprises
e) washing the solid containing 2,5-furandicarboxylic acid with a first washing solution comprising a saturated organic acid solvent having from 2 to 6 carbon atoms and less than 15% by weight of water, based on total amount of washing solution, preferably followed by washing with a second washing solution comprising water in an amount of more than 95% by weight with respect to the weight of the washing solution.

9. The process according to claim 8, which process further comprises
f) contacting washed solid containing 2,5-furandicarboxylic acid with polar solvent to obtain a solution;
g) contacting the solution with hydrogen in the presence of a hydrogenation catalyst at hydrogenation conditions yielding a hydrogenated solution; and
h) separating purified 2,5-furandicarboxylic acid from the hydrogenated solution.
